(19) United States
(12) Patent Application Publication
(10) Pub. No.: US 2014/0288270 A1
(43) Pub. Date: Sep. 25, 2014

(54) ISOLATED CHROMOPROTEIN OF STICHODACTYLA HADDONI

(71) Applicant: National Taiwan University, Taipei (TW)

(72) Inventors: Huai-Jen Tsai, Taipei (TW); Cheng-Yi Chiang, Taipei (TW); Yi-Lin Chen, Taipei (TW); Yen-Ting Chen, Taipei (TW)

(73) Assignee: National Taiwan University, Taipei (TW)

(21) Appl. No.: 14/011,393

(22) Filed: Aug. 27, 2013

(30) Foreign Application Priority Data
Mar. 22, 2013 (TW) 102110290

(51) Int. Cl. C07K 14/435

(52) U.S. Cl. CPC C07K 14/43595
USPC 530/350; 536/23.51; 435/320.1; 435/252.33

(57) ABSTRACT

The present application provides a chromoprotein (shCP), comprising amino acid sequences with greater than 96% consistency of SEQ ID NO: 1. The chromoprotein is derived from Stichodactyla haddoni and has an absorption spectrum of 350˜650 nm. The present application also provides a nucleic acid sequence, comprising a nucleic acid sequence encoding the amino acid sequence of the shCP. The present application further provides a vector, comprising the nucleic acid sequence of the shCP. The present application still provides a host, including the vector carried with the nucleic acid sequence of the shCP.

### FIELD OF THE INVENTION

An isolated chromoprotein of Stichodactyla haddoni.

### DESCRIPTION OF PRIOR ART

For traditional aquarium fish breeding, it is general using mating or hybridization to obtain a strain with a colorful or unique shape body. However, the number of high-quality strains is very rare. The lines and exterior features of the high-quality strains are difficult to be genetically preserved or extensive multiplied. Currently, biomolecular technology is applied to search novel chromoprotein genes from the colorful marine organisms. However, current widely used chromoproteins mostly are green or red fluorescent proteins. The blue or purple fluorescent proteins or chromoproteins are rarely being published, applied and patented. Based on the demand of the industry, the present invention isolates a novel purple chromoprotein from marine organisms which has novelty and industrial utility.

### BRIEF DESCRIPTION OF THE DRAWINGS

FIG. 1 shows light purple colonies of E. coli BL21 which expresses shCP chromoprotein.

FIG. 2 shows chromoproteins of purified shCP, shCP-Y64L/I196H, shCP-E63S, shCP-Q39S and shCP-T194I chromoprotein.

FIG. 3 shows the absorption spectra of (a) shCP, (b) shCP-Y64L/I196H, (c) shCP-E63S, (d) shCP-Q39S and (e) shCP-T194I.

FIG. 4 shows wild type Golden Zebra Danio and Golden Zebra Danio expressing shCP, shCP-I196T and shCP-E63S chromoprotein.

### SUMMARY OF THE INVENTION

The present invention provides a chromoprotein, Stichodactyla haddoni chromoprotein (shCP), comprising an amino acid sequence having at least 96% identity to SEQ ID NO: 1, wherein a position of mutant residue is at 39^{th}, 63^{th}˜64^{th}, 194^{th} or 196^{th} from the N-end of the descripted amino acid sequence. The shCP is isolated from a Stichodactyla haddoni, and a mutant residue of the SEQ ID NO: 1 is Q39S, E63S, Y64L, T194I or I196H. In a preferred embodiment of present invention, the amino acid sequence of shCP is SEQ ID NO: 1, SEQ ID NO: 2, SEQ ID NO: 3, SEQ ID NO: 4 or SEQ ID NO: 5. Furthermore, the shCP has an absorption spectrum from 350 to 650 nm.

The present invention also provides a labeling kit, which comprises a bio-labeling molecular, shCP. The present invention further provides a nucleic acid sequence, which encodes the amino acid sequence of shCP.

### DETAILED DESCRIPTION OF THE INVENTION

The present invention provides a chromoprotein, Stichodactyla haddoni chromoprotein (shCP), isolated from Stichodactyla haddoni. Site-directed and random mutagenesis are utilized to obtain mutated sequences encoding chromoproteins with various colors and E. coli and zebrafish are used as expressing systems to successfully express the colors of the isolated protein as well as the mutated amino acid sequences, providing evidences that chromoprotein can be expressed in prokaryotes and eukaryotes. The present invention can be applied on labeling with colors in biological research or on breeding of animal strains.

The present invention provides a chromoprotein, shCP, containing an amino acid sequence with higher than 96% of similarity to SEQ ID NO:1. This chromoprotein is isolated from Stichodactyla haddoni. The absorption spectrum of the chromoprotein ranges from 350 nm to 650 nm. In one embodiment, the chromoprotein is an artificially synthesized amino acid sequence.

In one embodiment, the mutable sites on the amino acid sequence of the chromoprotein are the 39^{th}, the 63^{th}, the 64^{th}, the 194^{th} and the 196^{th} amino acids. In a better embodiment, the 39^{th} amino acid is mutated from Q to S, the 63^{th} amino acid is mutated from E to S, the 64^{th} amino acid is mutated from Y to L, the 194^{th} amino acid is mutated from T to I or the 196^{th} amino acid is mutated from I to H. In another better embodiment, the chromoprotein is shCP, shCP-Y64L/I196H, shCP-E63S, shCP-Q39S or shCP-T194I with their corresponding amino acid sequences of SEQ ID NO: 1, SEQ ID NO: 2, SEQ ID NO: 3, SEQ ID NO: 4 or SEQ ID NO: 5.

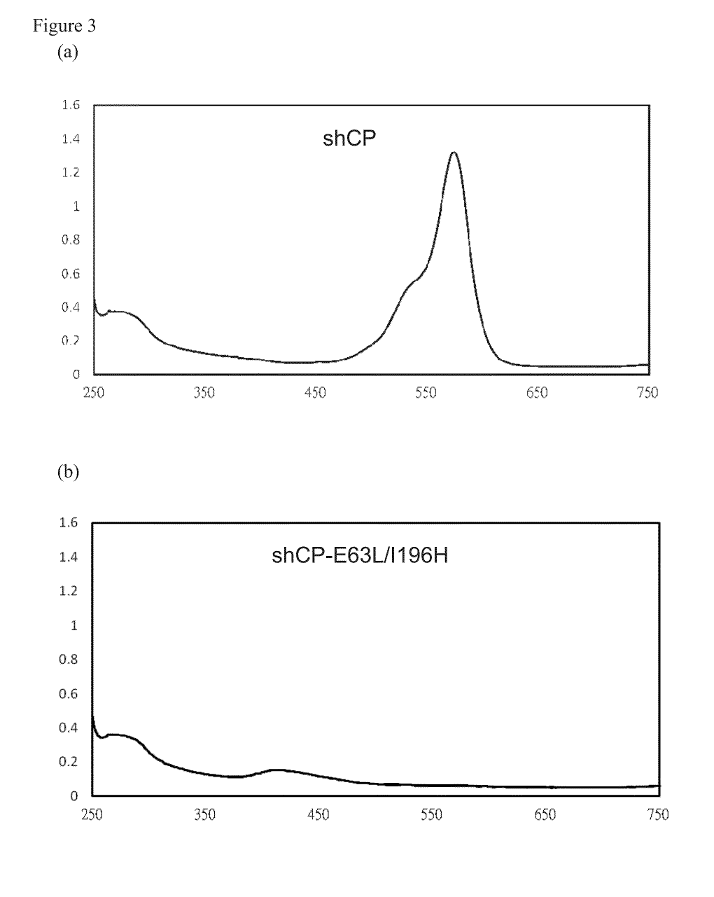

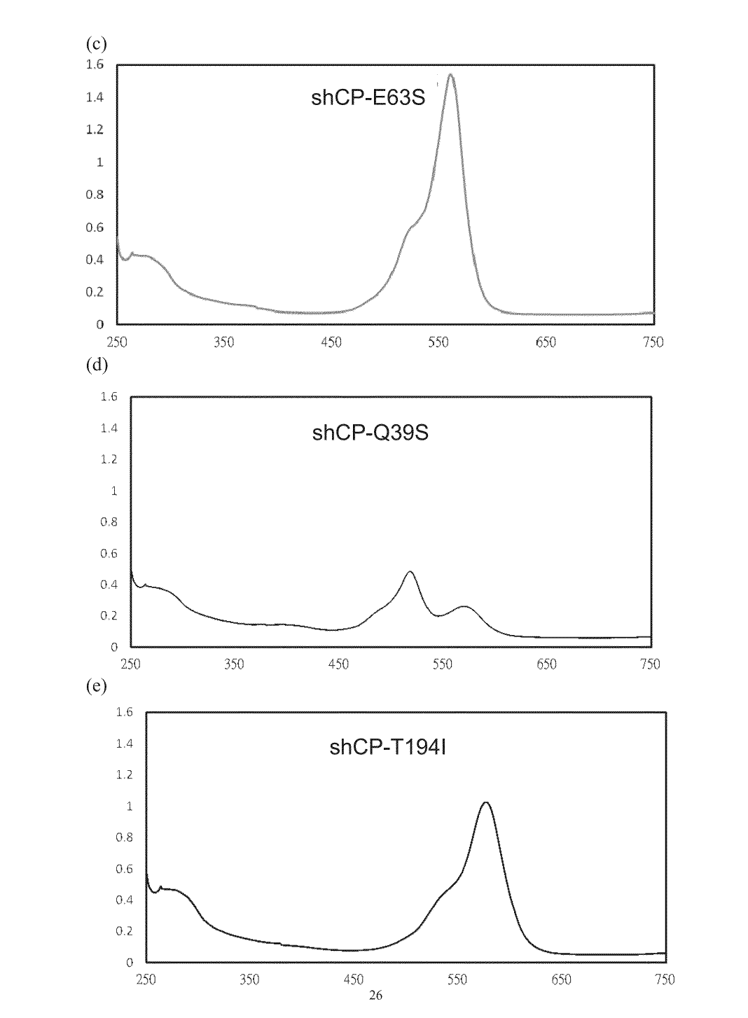

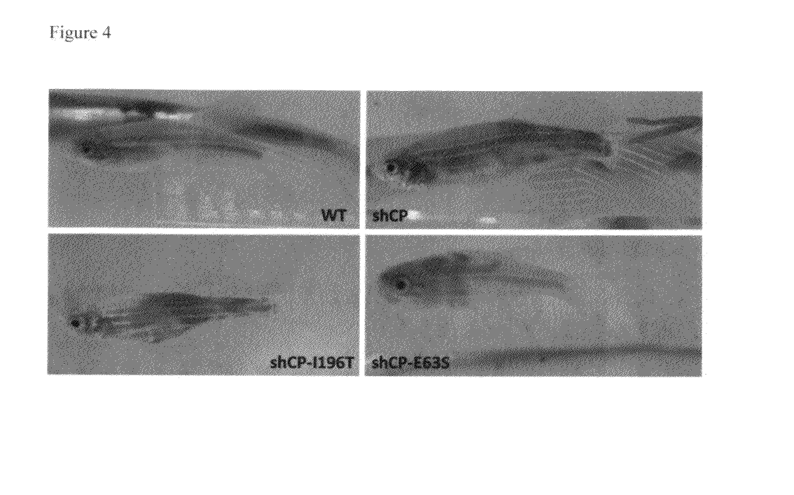

In one embodiment, the absorption spectrum of shCP ranges from 480 nm to 630 nm with its absorption peak at 574 nm (FIG. 3a); the absorption spectrum of shCP-Y64L/I196H ranges from 350 nm to 450 nm with its absorption peak at 415 nm (FIG. 3b); the absorption spectrum of shCP-E63S ranges from 460 nm to 600 nm with its absorption peak at 560 nm (FIG. 3c); the absorption spectrum of shCP-Q39S ranges from 450 nm to 600 nm with its absorption peak at 518 nm (FIG. 3d); the absorption spectrum of shCP-T194I ranges from 470 nm to 630 nm with its absorption peak at 577 nm (FIG. 3e).

The present invention provides a labeling kit containing a bio-labeling molecule, the shCP chromoprotein.

The present invention also provides a nucleotide sequence encoding the amino acid sequence of shCP chromoprotein. The present invention further provides a carrier, containing the nucleotide sequence of the shCP chromoprotein. The present invention also provides a host containing the carrier carrying the nucleotide sequence of the shCP chromoprotein, wherein the carrier could be a vector.

In one embodiment, the nucleotide sequence of the shCP chromoprotein is an artificially synthesized nucleotide sequence. In another embodiment, the host is a eukaryote or a prokaryote. In a better embodiment, the hosts include, but not limited to, prokaryotic unicellular organisms, eukaryotic unicellular organisms, fish or mammalians.

In one embodiment, the chromoprotein in the present invention is utilized for breeding of aquarium fish. In yet another embodiment, the chromoprotein in the present invention can be used as an additive in foods or medicine.

### EXAMPLES

### Example 1

The present invention selected Stichodactyla haddoni, which had no chormoprotein sequence published, as the object for searching a chromoprotein. First, glass beads were applied to ground the tentacle tissue cells. A raw purified total protein solution was isolated. A chromatic band of the chromoprotein was separated from the Stichodactyla haddoni total protein solution by using native PAGE. The chromatic band of the chromoprotein was cut out from the native-PATE, and further separated using SDS-PAGE. The molecular weight (MW) of the separated chromoprotein monomer was about 25 kDa. The chromoprotein monomer with MW of 25 kDa was further analized using Liquid Spectrometer Mass Chromatograph (LCMS-MS) to obtain the amino acid sequence of Stichodactyla haddoni chromoprotein (SEQ ID No: 1).

cDNA Synthesis

3.5 μl of total RNA (0.5-1 μg) isolated from Stichodactyla haddoni was taken and was added in 1 μl dT(15)-T7 primer. The reaction was at 70° C. for 3 minutes, followed by 2 minutes of reaction at 42° C. 2 μl of 5× First-Strand Buffer, 0.25 μl of 100 mM DTT, 1 μl of 10 mM dNTP mixture, 1 μl of 10 mM TS primer, 0.25 μl of RNase inhibitor and 1 μl of Superscript III reverse transcriptase were then added and reacted for 90 minutes at 42° C., followed by 10 minutes at 68° C. to terminate the reaction in order to yield a cDNA library of Stichodactyla haddoni. Then, partial region of the chromoprotein derived from analysis was used for designing degenerated primer. Rapid amplification of cDNA ends (RACE) was utilized to search for gene of Stichodactyla haddoni chromoprotein.

Rapid Amplification of cDNA Ends, RACE

PCR was used for preparation for particular cDNA fragment. PCR reaction solution was prepared by adding 31.5 μl of DD water, 10 ρl of hifi 5× buffer (Kapa Biosystems), 4 μl of 2.5 μg/ml dNTP, 2 μl of Stichodactyla haddoni cDNA template, 1 μl of 10 mM TS primer or dT(15)-T7 primer and 1 μl of 10 mM degenerated primer for 5 minutes reaction at 94° C. 0.5 μl of hifi DNA polymerase (Kapa Biosystems) was then added at 80° C. After that, a cycle of 30-second reaction at 94° C. , 30-second reaction at 60° C. and then 30-second reaction at 72° C. was repeated 230 times, followed by 10 minutes of reaction at 72° C. The PCR fragments were then purified and dissolved using 30 μl of DD water, followed by elimination of residual solvent and enzyme using DNA Clean up kit (Geneaid).

In this example, using the cDNA library of Stichodactyla haddoni, 3′-RACE and 5′-RACE were proceeded by following the degenerated primer to yield a close-to-300 bp and a close-to-400 bp DNA sequences respectively. The two sequences were individually cloned, sequenced and compared to obtain the complete cDNA sequence of Stichodactyla haddoni chromoprotein.

Site-directed and random mutagenesis were performed on gene of the Stichodactyla haddoni chromoprotein obtained. It was found 5 sequences including shCP (SEQ ID No: 1), shCP-Y64L/I196H (SEQ ID No: 2), shCP-E63S (SEQ ID No: 3), shCP-Q39S (SEQ ID No: 4) and shCP-T194I (SEQ ID No: 5) express colors different from each other. The present invention further utilized E. coli expressing system and zebrafish expressing system to prove that the chromoprotein and its mutants could be expressed in prokaryotes and eukaryotes.

### Example 2

### E. Coli Transformation

1. Transformation:

10 μl of competent cells at 4° C. (DH5α, JM106 and BL21) were mixed with 1 μl of plasmid, and then placed on ice for 20 minutes. After that, the mixture was incubated in water bath at 42° C. for 1 minute, followed by 3 minutes of incubation on ice. The culture was then spread onto LB plates containing 50 μg/ml of ampicillin and incubated for 16 hours at 37° C.

2. Ligation Transformation:

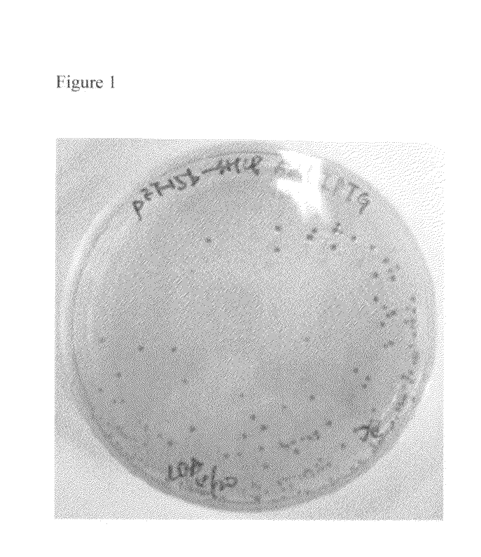

100 μl of competent cells were mixed with 10 μl of ligation solution, and then placed on ice for 20 minutes. After that, the mixture was incubated in water bath at 42° C. for 1 minute, followed by 3 minutes of incubation on ice. 1 ml of LB was then added and the mixture is incubated for 1 hour at 37° C., followed by 5 minutes of centrifugation at 8000 rpm to eliminate most of the supernatant. The culture was then spread onto LB plates containing 50 μg/ml of ampicillin and incubated for 16 hours at 37° C. The transformed BL21 E. coli was added in IPTG to induce expression of the protein. After 24 hours, colonies expressing light violet shCP could be observed. After 72 hours at 4° C., massive accumulation of shCP could be observed (as shown in FIG. 1).

Expression and Purification of Protein from E. Coli

1. Isolation of E. Coli-Expressed Protein

BL 21 E. coli was transfected with plasmids of pET-shCPM, pET-shCP-E63S, pET-shCP-Q39S, pET-shCP-T194I or pET-shCP-Y64Y/I194H and cultured for 16 hours with 3 ml of LB containing 50 μg/ml of ampicillin. The E. coli cultures were then diluted 100-fold and incubated at 37° C. for 3-5 hours till the OD600 value reaches 0.4-0.5, followed by addition of IPTG (final concentration: 1 mM) to induce protein expression. The E. coli cultures were incubated at 20° C. till significant color appears. Depending upon various mutant proteins, the incubation requires approximately 2 to 4 days. The cultures were then centrifuged at 8 krpm, and the supernatant was discarded. 2 ml of binding buffer (10 mM NaH₂PO₄, 10 mM Na₂HPO₄, 500 mM NaCl and 20 mM imidazole pH7.4) was then added and the E. coli was resuspended. The E. coli cultures were placed on ice and sonicated for 10-15 minutes, followed by 20 minutes of centrifugation at 13.2 krpm. The supernatants were collected and from which the chromomprotein was purified by using Ni-NTA Spin Kit (GE).

2. Purification of E. Coli-Expressed Protein

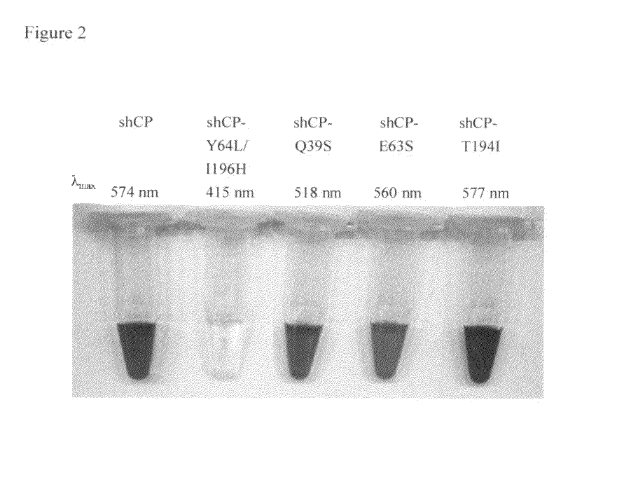

Binding buffer and elution buffer (10 mM NaH₂PO₄, 10 mM Na₂HPO₄, 500 mM NaCl and 500 mM imidazole pH7.4) were prepared. 10 ml of binding buffer was added into the purification column (GE) to equilibrate the buffer solution, then the E. coli protein extract was added into the column. Protein was separated by gravity. 10 ml of binding buffer was added into the purification column to eliminate the his-tag-free protein. The step above was repeated twice. Then, 3 ml of elution buffer was added into the column. The colored solution dropping out was the purified protein. After protein purification, HiTrap Desalting (GE) and desalting buffer (10 mM NaH₂PO₄, 10 mM Na₂HPO₄ and 500 mM NaCl) were used to eliminate imidazole. Purified shCP, shCP-Y64L/I196H, shCP-E63S, shCP-Q39S and shCP-T194I chromoproteins were shown in FIG. 2.

Spectrophotometric Protein Assay:

Pierce 660 nm Protein Assay Kit was used to measure the concentration of the purified protein. Below 0.25 mg/ml of protein concentration, a spectrophotometer (Beckman DU640B) with 1 cm quartz cuvette was used to measure the absorption spectrum, ranging from 250 nm to 800 nm, of the chromoproteins, including shCP, shCP-Y64L/I196H, shCP-E63S, shCP-Q39S and shCP-T194I. A fluorescence sprctrometer (Hitachi F-7000) was used to measure the excitation and emission spectrums. The results were shown in FIG. 3. The absorption spectrum of shCP ranged from 480 nm to 630 nm with its absorption peak at 574 nm (FIG. 3a); the absorption spectrum of shCP-Y64L/I196H ranged from 350 nm to 450 nm with its absorption peak at 415 nm (FIG. 3b); the absorption spectrum of shCP-E63S ranged from 460 nm to 600 nm with its absorption peak at 560 nm (FIG. 3c); the absorption spectrum of shCP-Q39S ranged from 450 nm to 600 nm with its absorption peak at 518 nm (FIG. 3d); the absorption spectrum of shCP-T194I ranged from 470 nm to 630 nm with its absorption peak at 577 nm (FIG. 3e).

While the invention has been described and exemplified in sufficient detail for those skilled in this art to make and use it, various alternatives, modifications, and improvements should be apparent without departing from the spirit and scope of the invention.

One skilled in the art readily appreciates that the present invention is well adapted to carry out the objects and obtain the ends and advantages mentioned, as well as those inherent therein. The detecting samples (such as cells or PMMA), the apparatus and processes and methods for producing them are representative of preferred embodiments, are exemplary, and are not intended as limitations on the scope of the invention. Modifications therein and other uses will occur to those skilled in the art. These modifications are encompassed within the spirit of the invention and are defined by the scope of the claims.

What is claimed is:

1. An isolated chromoprotein comprising an amino acid sequence having at least 96% identity to SEQ ID NO: 1, wherein a position of mutable residue is at 39^{th}, 63^{th}˜64^{th}, 194^{th} or 196^{th} from the N-end of the said amino acid sequence.

2. The chromoprotein of claim 1, which is isolated from a Stichodactyla haddoni.

3. The chromoprotein of claim 1, wherein a mutant residue of the SEQ ID NO: 1 is Q39S, E63S, Y64L, T194I or I196H.

4. The chromoprotein of claim 1, wherein the amino acid sequence is SEQ ID NO: 1, SEQ ID NO: 2, SEQ ID NO: 3, SEQ ID NO: 4 or SEQ ID NO: 5.

5. The chromoprotein of claim 1, which has an absorption spectrum from 350 to 650 nm.

6. The chromoprotein of claim 1, which is an artificial synthesized amino acid sequence.

7. A labeling kit comprising a bio-label molecular which is the chromoprotein of claim 1.

8. A nucleic acid sequence which encodes the amino acid sequence of the chromoprotein of claim 1.

9. A vector comprising the nucleic acid sequence of claim 8.

10. A host comprising the vector of claim 9.

11. The nucleic acid sequence of claim 8, which is an artificial synthesized nucleic acid sequence.


SEQUENCE LISTING (5 sequences)

SEQ ID NO: 1 - protein, 227 aa, Stichodactyla haddoni
  PEPTIDE (1)..(227)
  PEPTIDE (1)..(227)
MAGLLKESMRIKMDMEGTVNGHYFKCEGEGDGNPFTGTQSMRIHVTEGAPLPFAFDILAPCCEYGSRTFIHHTAGIPDFFKQSFPEGFTWERTTTYEDGGILTAHQDTSLEGNCLIYKVKVLGTNFPADGPVMKNKSEGWEPCTEVVYPDNGVLCGRNVMALKVGDRRLICHLYSSYKSKKAIRALTMPGFHFTDIRLQMPRKKKDEYFELYEASVARYSDLPEKAN

SEQ ID NO: 2 - protein, 227 aa, Stichodactyla haddoni
  PEPTIDE (1)..(227)
MAGLLKESMRIKMDMEGTVNGHYFKCEGEGDGNPFTGTQSMRIHVTEGAPLPFAFDILAPCCELGSRTFIHHTAGIPDFFKQSFPEGFTWERTTTYEDGGILTAHQDTSLEGNCLIYKVKVLGTNFPADGPVMKNKSEGWEPCTEVVYPDNGVLCGRNVMALKVGDRRLICHLYSSYKSKKAIRALTMPGFHFTDHRLQMPRKKKDEYFELYEASVARYSDLPEKAN

SEQ ID NO: 3 - protein, 227 aa, Stichodactyla haddoni
  PEPTIDE (1)..(227)
MAGLLKESMRIKMDMEGTVNGHYFKCEGEGDGNPFTGTQSMRIHVTEGAPLPFAFDILAPCCSYGSRTFIHHTAGIPDFFKQSFPEGFTWERTTTYEDGGILTAHQDTSLEGNCLIYKVKVLGTNFPADGPVMKNKSEGWEPCTEVVYPDNGVLCGRNVMALKVGDRRLICHLYSSYKSKKAIRALTMPGFHFTDIRLQMPRKKKDEYFELYEASVARYSDLPEKAN

SEQ ID NO: 4 - protein, 227 aa, Stichodactyla haddoni
  PEPTIDE (1)..(227)
MAGLLKESMRIKMDMEGTVNGHYFKCEGEGDGNPFTGTSSMRIHVTEGAPLPFAFDILAPCCEYGSRTFIHHTAGIPDFFKQSFPEGFTWERTTTYEDGGILTAHQDTSLEGNCLIYKVKVLGTNFPADGPVMKNKSEGWEPCTEVVYPDNGVLCGRNVMALKVGDRRLICHLYSSYKSKKAIRALTMPGFHFTDIRLQMPRKKKDEYFELYEASVARYSDLPEKAN

SEQ ID NO: 5 - protein, 227 aa, Stichodactyla haddoni
  PEPTIDE (1)..(227)
MAGLLKESMRIKMDMEGTVNGHYFKCEGEGDGNPFTGTQSMRIHVTEGAPLPFAFDILAPCCEYGSRTFIHHTAGIPDFFKQSFPEGFTWERTTTYEDGGILTAHQDTSLEGNCLIYKVKVLGTNFPADGPVMKNKSEGWEPCTEVVYPDNGVLCGRNVMALKVGDRRLICHLYSSYKSKKAIRALTMPGFHFIDIRLQMPRKKKDEYFELYEASVARYSDLPEKAN